(19) United States
(12) Patent Application Publication
(10) Pub. No.: US 2026/0259219 A1
(43) Pub. Date: Sep. 3, 2026

(54) MEASURING REDOX CHANGES IN PLASMA PROTEINS

(71) Applicant: Cancer Research Technology Limited, London (GB)

(72) Inventors: Sara Rossana Zanivan, London (GB); Sergio Lilla, London (GB); Ulla-Maja Hagbo Bailey, London (GB)

(73) Assignee: Cancer Research Technology Limited, London (GB)

(21) Appl. No.: 19/164,019

(22) PCT Filed: Mar. 17, 2023
(86) PCT No.: PCT/EP2023/056974

§ 371 Date: Sep. 10, 2025

(51) Int. Cl. G01N 33/68; G01N 1/44; G01N 33/58; G16H 50/30

(52) U.S. Cl. CPC G01N 33/6848; G01N 1/44; G01N 33/58; G16H 50/30; G01N 2440/20; G01N 2560/00; G01N 2570/00; G01N 2800/50

(57) ABSTRACT

Provided is a method for measuring proteomic cysteine redox status in a sample obtained from a bodily fluid of a subject, the method comprising: (i) providing said sample, wherein the sample comprises a plurality of different proteins that comprise cysteine residues, a proportion of which are in reduced form and a proportion of which are in reversibly oxidised form; (ii) contacting the sample with a first label that selectively labels the reduced cysteine thiols of the plurality of different proteins; (iii) contacting the sample with a reducing agent that reduces the reversibly oxidised cysteine residues to form reduced cysteine thiols; (iv) contacting the sample with a second label that labels the reduced cysteine thiols formed in step (iii), wherein the second label is distinguishable from the first label by mass spectrometry, (v) processing the sample for analysis by mass spectrometry; and (vi) analysing the sample by mass spectrometry to measure at least the relative abundance of identified peptides labelled with the first label and corresponding identified peptides labelled with the second label, thereby providing a measure of the proteomic cysteine redox status of the sample. The invention also provides related methods for biomarker discovery and for predicting presence of a liver disease and/or cancer, such as hepatocellular carcinoma (HCC) in a subject.

### FIELD OF THE DISCLOSURE

The present disclosure relates to methods for measuring redox changes at cysteine residues of proteins in plasma samples, and the use of such methods for detecting disease, for example liver cirrhosis and hepatocellular carcinoma (HCC).

### BACKGROUND TO THE INVENTION

Thiol groups (—SH) of cysteine residues undergo a broad variety of oxidative modifications in cells, such as sulfenylation, nitrosylation, and glutathionylation. Such modifications alter the ratio of reduced cysteine residues to oxidised cysteine residues. These changes are particularly apparent in disease states, where cells experience oxidative stress due to an increase in reactive oxygen species (ROS), causing increased oxidation of thiol groups. High levels of ROS have been implicated in the pathology of numerous diseases, including cancer, Alzheimer's disease, Parkinson's disease, diabetes mellitus, atherosclerosis, and many inflammatory diseases. Oxidative changes may be reversible, such as glutathionylation or oxidation to sulfenic acid (—SOH), or irreversible, such as oxidation to sulfonic acid (—SO₃H). Reversible oxidation is a common regulatory mechanism used for example to modulate proteins and enzymes that are themselves involved in the response to oxidative stress, whereas irreversible oxidation leads to protein dysfunction and is a hallmark of oxidative stress.

Hepatocellular carcinoma (HCC) does not usually cause symptoms in early stages. At present, most HCC patients are diagnosed at an advanced, often incurable, stage. Conversely, if HCC is detected in its early stages, surgery may be curative. Identifying early stage HCC among a patient population having liver cirrhosis is particularly challenging. Cirrhosis patients may be monitored, e.g., every 6 months by ultrasound and measurement of blood levels of tumour marker alpha-fetoprotein (ΔFP). Elevated levels of AFP are associated with HCC, but this biomarker can be unreliable. When screening indicates the presence of HCC, follow-up diagnosis and staging may be performed using MRI and CT scans.

Redox proteomics involves established methods of labelling cysteine residues to identify redox changes on a whole-proteome scale. Cysteines are labelled to preserve the endogenous oxidation state of their thiol group, using labels which can then be identified using mass spectrometry or related techniques. Iodoacetamide (IAA) and N-ethylmaleimide (NEM) are commonly used as labels in redox proteomics, which both react with free reduced thiol groups. IAA reacts via nucleophilic substitution, wherein the thiolate functions as the nucleophile and the iodine as the leaving group. Conversely, NEM is a Michael acceptor, and reacts via Michael-type addition.

Redox proteomics can be used to detect changes in redox status that indicate disease. Proteins may be isolated from the diseased tissue itself, or from other bodily fluids such as the blood. Using blood samples for redox proteomics is advantageous, as this is a routine and non-invasive method. However, blood samples provide a systemic report, and cannot provide as much information as compared to samples taken directly from the diseased tissue, such as e.g. a tumour sample. The protein fraction of blood plasma consists of 50-60% albumin, and 40% globulins, leaving tissue-specific proteins present in very low amounts. This means that when performing redox proteomics on blood samples, the majority of peptides will have derived from albumin or globulins, and a highly sensitive test would be required to identify redox changes on the remaining tissue-specific peptides. To overcome this problem, enrichment steps can be performed to increase the proportion of cysteine-containing peptides. Such steps require extensive manipulation of samples, however, increasing the chance of sample loss and/or contamination during sample preparation. Most importantly though, specific or insufficient enrichment steps may introduce bias, affecting the amounts of proteins present in the final sample and confounding any indication of disease.

Therefore, an unmet need exists for improved methods enabling the detection of redox changes in blood samples using a redox proteomics approach. The present invention aims to address these unmet needs and provide related advantages.

### SUMMARY OF THE INVENTION

Broadly, the present inventors hypothesised that a method which is able to measure global protein oxidation of, e.g., hundreds of proteins from a readily accessible sample source would provide greater insight into health and disease states than prior described methods that, for example, measure only one or two proteins such as serum albumin. In particular, cysteines at different sites on different proteins may exhibit different propensity for oxidation, reflecting their different sources (e.g. liver-derived proteins vs. immunoglobulins from immune cells). Through diligent research the present inventors surprisingly found that they were able to apply a double-labelling approach for measurement of cysteine oxidation to bodily fluid samples, in particular plasma samples. As described in the examples herein, the method of the present invention demonstrates the identification of peptide redox biomarkers of early-stage HCC as compared to both liver cirrhosis patients and healthy subjects.

Accordingly, in a first aspect the present invention provides a method for measuring proteomic cysteine redox status in a sample obtained from bodily fluid of a subject, the method comprising:
(i) providing said sample, wherein the sample comprises a plurality of different proteins that comprise cysteine residues, a proportion of which are in reduced form and a proportion of which are in reversibly oxidised form;
(ii) contacting the sample with a first label that selectively labels the reduced cysteine thiols of the plurality of different proteins;
(iii) contacting the sample with a reducing agent that reduces the reversibly oxidised cysteine residues to form reduced cysteine thiols;
(iv) contacting the sample with a second label that labels the reduced cysteine thiols formed in step (iii), wherein the second label is distinguishable from the first label by mass spectrometry,
(v) processing the sample for analysis by mass spectrometry; and
(vi) analysing the sample by mass spectrometry to measure at least the relative ab undance of identified peptides labelled with the first label and corresponding identified peptides labelled with the second label, thereby providing a measure of the proteomic cysteine redox status of the sample.

In certain embodiments the sample has been obtained from a circulating bodily fluid, such as a plasma sample, a blood sample, a circulating exosome-containing liquid sample, a cerebrospinal fluid sample and a lymphatic fluid sample. In some embodiments the sample has been obtained from non-circulating bodily fluids such as ascites or pleural effusion.

In certain embodiments the sample is heated to a temperature of greater than 50° C., optionally greater than 90° C., for a period of time sufficient to increase protein denaturation prior to or current with contacting the sample with said first label. Heating the sample in this way has been found to improve labelling of cysteine residues allowing great capture of the proteome redox signal.

In certain embodiments the first label is selected from: iodoacetamide-light (“IAA-light”), n-ethylmaleimide (NEM) and heavy stable isotope-labelled iodoacetamide (“IAA-heavy”). Likewise, the said second label may be selected from: IAA-heavy, IAA-light and n-ethylmaleimide (NEM), provided that said second label differs from said first label. In certain embodiments, the first label comprises IAA-light and said second label comprises IAA-heavy.

In certain embodiments the reducing agent comprises dithiothreitol (DTT).

In certain embodiments the sample is solubilised by contacting with sodium deoxycholate (SDC) prior to or during step (ii). Use of SDC to solubilise the sample has been found to increase the performance of the method by increasing the proportion of cysteine residues that are labelled.

In certain embodiments the sample is contacted with magnetic beads, optionally SpeedBead Magnetic Carboxylate (SP3 beads), prior to or during step (v). The bead-based processing has been found to improve trypsin digestion of the proteins from the sample and thereby enhance the performance of the method.

In certain embodiments the method further comprises contacting the sample with at least one tandem mass tag (TMT) to enable multiplexing of said mass spectrometry analysis.

In certain embodiments the concentration of said second label in step (iv) is higher than the concentration of said first label in step (ii). For example, where the first label comprises IAA-light it may be at a concentration during labelling of between 0.015M and 0.025M, optionally at 0.02M, and where the second label comprises IAA-heavy it may be at a concentration during labelling of between 0.03M and 0.05M, optionally at 0.04M.

In certain embodiments the reducing agent comprises DTT at a concentration during the reducing step of between 0.025M and 0.045M, optionally at 0.033M.

In certain embodiments the processing of step (v) comprises at least one of: enzymatic digestion of the proteins in the sample (e.g. trypsin digestion), acidification of the sample, and peptide fractionation of the sample.

In certain embodiments the mass spectrometry (MS) comprises ultra-high performance liquid chromatography-MS/MS (UHPLC-MS/MS).

In certain embodiments step (vi) further comprises calculating, for each of a plurality of cysteine residues, the proportion that was in reversibly oxidised form in the sample.

In certain embodiments step (vi) further comprises deriving at least one statistical measure, optionally a z-score, for the degree of cysteine oxidation of each of a plurality of non-identical peptides.

In certain embodiments the plurality of different proteins comprises at least 50, 100, 500, or at least 1000 different proteins.

In certain embodiments the sample is of less than 100 μl, less than 50 μl, less than 10 μl, less than 1 μl or less than 100 nl when obtained from the subject. The present inventors have found that the SICyLIA method is very sensitive and is able to derive proteome redox signal from extremely small amounts of sample (such as 1 μl of plasma or less). This makes the method well-suited to non-invasive testing, including repeated testing of the same subject over time.

In certain embodiments the method further comprises normalising the measure of identified peptides labelled with the first label and/or the measure of identified peptides labelled with the second label for protein abundance. Where the redox status is determined as the ratio between reduced cysteines and reversibly oxidised cysteines for the same peptide from the same sample, normalisation by protein or peptide amount may not be necessary. However, in certain situations normalising to account for different protein amounts may be advantageous. The normalising may comprise normalising for the level of the corresponding cysteine-containing protein in the sample as determined by mass spectrometry.

In a second aspect the present invention provides a method for identifying biomarkers that are significantly associated with a particular disease state, the method comprising:
performing the method of the first aspect of the invention on a plurality of samples obtained from subjects known not to have the particular disease state (“controls”) and on a plurality of samples obtained from subjects known to have the particular disease state (“diseased subjects”) and thereby determining the proteomic cysteine redox status of the controls and the diseased subjects for a plurality of proteins and/or peptides;
comparing the proteomic cysteine redox status of the controls and the diseased subjects to identify proteins and/or peptides that are significantly oxidised or reduced in the samples from the diseased subjects as compared with the samples from the controls, thereby identifying these proteins and/or peptides as biomarkers of the particular disease state.

In certain embodiments the subjects known not to have the particular disease state have a different disease or have the same disease as the particular disease state, but are at a different disease stage. This is the case where the method is for discriminating between disease states (including closely related or adjacent disease states).

In certain embodiments the subjects known not to have the particular disease state are healthy controls.

In certain embodiments the particular disease state may be a liver disease and/or a cancer. For example, the particular disease state may be early-stage hepatocellular carcinoma (HCC).

In certain embodiments the subjects known not to have the particular disease state are subjects who are known to have liver cirrhosis without HCC; are subjects who are known to have hepatitis B without HCC; and/or are subjects who are known to have hepatitis C without HCC.

In certain embodiments the particular disease state is a metastatic cancer of known primary and the subjects known not to have the particular disease state have a cancer, optionally a metastatic cancer, of a primary source that differs from said known primary of the particular disease state. In this way the method may be used to identify proteome redox biomarkers that are informative for determining a cancer of unknown primary (CUP).

In still further embodiments, the method may be for identifying biomarkers for disease monitoring, such as monitoring disease response to therapy (e.g. if a proteome redox signature characteristic of a tumour remains present, the disease has not responded to the therapy; if the proteome redox signature characteristic of a tumour decreases over time or is absent, the disease had responded to the therapy). Furthermore, the method of the present invention may be employed to predict response to a treatment. Where a proteome redox signature is characteristic of a particular tumour status (e.g. informative of the genetic status of the tumour and/or the immune status of the tumour), this may be used to inform treatment strategy.

In a third aspect the present invention provides a method for determining that a subject is likely to have hepatocellular carcinoma (HCC), optionally early-stage HCC, the method comprising:
performing the method of the first aspect of the invention on a bodily fluid sample obtained from the subject thereby determining the proteomic cysteine redox status of the sample for a plurality of proteins and/or peptides;
providing data that represents the proteomic cysteine redox status of the sample for the plurality of proteins and/or peptides to a machine learning classifier, wherein said classifier has been trained on a training dataset that comprises proteomic cysteine redox status for the same plurality of proteins and/or peptides from both subjects known to have HCC and subjects known not to have HCC;
causing the machine learning classifier to classify the sample as belonging to the HCC class or not the HCC class based on the provided proteomic cysteine redox status of the sample; and
optionally, outputting the classification to a user.

In certain embodiments the subject has, or is suspected to have, liver cirrhosis.

In certain embodiments the machine learning classifier has been trained on a training data set that comprises proteomic cysteine redox status from subjects known to have early-stage HCC and subjects known to have liver cirrhosis without HCC.

In certain embodiments the machine learning classifier comprises a processor and non-transitory memory comprising instructions that when run by the processor cause the processor to process the data representing the proteomic cysteine redox status of the sample for the plurality of proteins and/or peptides according to a learnt model and to output a probabilistic classification of HCC or non-HCC.

In certain embodiments the learnt model is selected from: a decision tree, a logistic regression model, an artificial neural network, a support vector machine (SVM), naïve bayes or k-nearest neighbour algorithm, optionally wherein the decision tree comprises a gradient boosting algorithm or a random forest algorithm.

In certain embodiments the plurality of proteins and/or peptides comprises at least 5, 10, 15 or at least 20 proteins, or cysteine-containing peptide fragments thereof, selected from: GC (P02774), ALB (P02768), IGFALS (P35858), SERPIND1 (P05546), AZGP1 (P25311), PLG (P00747), IGKC (P01834), CD5L (043866), TF (P02787), C7 (P10643), IGHG1 (P01857), CFB (P00751), IGHG3 (P01860), APOB (P04114), SERPINC1 (P01008), F2 (P00734), C1S (P09871), CP (P00450)), APOH (P02749), AMBP (P02760) and HP (P00738). The identifier in brackets after each protein/gene name being the UniProt accession number for the protein in question as of UniProt release 2023_01 (release date 22 Feb. 2023).

In certain embodiments the plurality of proteins and/or peptides comprises at least 5, 10, 15, 20 or even all of the peptides shown in FIG. 11 (SEQ ID NOs: 114-152).

In certain embodiments the plurality of proteins and/or peptides comprise at least 5, 10, 15 or at least 20 of the peptides of SEQ ID NOs: 1 to 113.

In certain embodiments the plurality of proteins and/or peptides comprise at least 5, 10, 15 or at least 20 of the peptides of SEQ ID NOs: 114 to 152.

In certain embodiments the machine learning classifier is also provided with one or more additional biomarker measurements, optionally protein abundance measurements, obtained from the sample, and wherein the machine learning classifier has been trained on a training data set that further comprises the one or more additional biomarker measurements from both subjects known to have HCC and subjects known not to have HCC. In particular, the one or more additional biomarker measurements mat comprise: the blood level of alpha-fetoprotein (AFP) and/or any one of the proteins shown in FIG. 10.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE DRAWINGS

FIG. 1 shows A) the chemical reaction between a free thiol group and IAA-light, which results in the addition of a carbamidomethyl (CAM) group to the cysteine residue, releasing I⁻ and H⁺, and B) the chemical structure of IAA-light and IAA-heavy, containing the stable isotopes ¹²C and H or ¹³C and deuterium, respectively;

FIG. 2 shows an example SICyLIA labelling strategy, wherein IAA-light and IAA-heavy are used to distinguish between reduced cysteine residues of a control and experimental sample, respectively. Using this approach, reversibly oxidised residues of both the control and experimental samples are reduced using DTT, and then labelled with NEM;

FIG. 3 shows plots of all the cysteine-containing peptides identified in the plasma samples analysed by mass spectrometry (MS), the percentage of those that were labelled with IAA (blue) and those that were not labelled with IAA (orange). A) SDC: Sodium deoxycholate solubilised samples and B) SDS: Sodium dodecyl sulfate solubilised samples.

FIG. 4 shows a bar graph illustrating the abundance of tissue-specific plasma proteins, excluding those which are derived from the blood (albumin and globulins). The data was obtained using the Human Protein Atlas;

FIG. 5 shows the results of boiling plasma samples before performing SICyLIA; A) a heatmap showing the abundance of specific peptides derived from albumin and labelled with IAA on cysteine residues, in boiled and non-boiled samples. Plasma samples were subject to SICyLIA using a dual labelling approach, wherein reduced cysteine residues were labelled with IAA-light and reversibly oxidised cysteine residues were labelled with NEM-light. The labelled samples were analysed by UHPLC-MS, and data analysis was performed using the MaxQuant software. The data are log₂ transformed, as output by the MAxQuant software. The data shows the quantification of the IAA-light label, i.e. the reduced cysteine residues; B) shows the overall number of different plasma peptides identified by IAA-light labelling and detected in boiled and non-boiled samples, and C) a heatmap showing the abundance of a number of different peptides, in boiled and non-boiled samples. The data shows quantification of IAA-light, i.e. reduced cysteine residues, presented as log₂ transformed;

FIG. 6 shows the percentages of fully cleaved peptides generated during trypsin digest, performed on peptides in solution or adsorbed to SP3 magnetic beads;

FIG. 7 shows a graphical representation of the evolution of oxidative stress during liver disease. Oxidative stress increases as liver disease progresses from cirrhosis to early HCC, and as a result the redox state of cysteine residues is shifted towards oxidised. These changes can be detected by measuring cysteine-containing liver proteins secreted into the bloodstream;

FIG. 8 provides A) an overview of the patients and donors used in the HCC redox proteomics study, B) the proportion of males and females in each patient class, and C) the spread of age groupings within each patient class (split by groupings to preserve anonymity);

FIG. 9 demonstrates the different redox environments found intracellularly and extracellularly. Plasma samples were subject to SICyLIA with dual labelling of both reduced and reversibly oxidised residues, and the number of peptides of each redox state were summed to calculate a percentage;

FIG. 10 shows a heatmap showing the differential abundance of proteins in healthy donors, patients with cirrhosis, and patients with early HCC. Plasma samples were subject to SICyLIA with dual labelling of reduced cysteine residues with IAA-light, and reversibly oxidised cysteine residues with NEM-light. Protein abundance values were processed to calculate Z-scores using the MaxQuant and Perseus software;

FIG. 11 shows a heatmap showing the differential redox status of peptides derived from specific proteins, in healthy donors, patients with cirrhosis, and patients with early HCC. Plasma samples were subject to SICyLIA with dual labelling of reduced cysteine residues with IAA-light, and reversibly oxidised cysteine residues with NEM-light. Z-scores were computed using Perseus, wherein-2 indicates increased or decreased oxidation of IAA-labelled or NEM-labelled peptides, respectively, and 2 indicates increased or decreased reduction of IAA-labelled or NEM-labelled peptides, respectively. The proteins from which the peptides were derived are indicated to the left of the heatmap using their respective gene names. Replicated gene names indicate different cysteine-containing peptides from the same protein.

FIG. 12 shows a graphical representation of a workflow that may be implemented to identify biomarkers and build a classifier based on differential redox changes detected using the SICyLIA in plasma methods. Plasma samples are collected from patients (as little as 1 μl in volume) and subjected to SICyLIA to label reduced and reversibly oxidised cysteine residues. Labelled samples are analysed using UHPLC-MS/MS, and the results are used to train a machine learning algorithm to classify individuals into certain patient groups based on the changes in redox state at certain cysteine residues. Advantageously, the classifier may discriminate adjacent disease states, such as liver cirrhosis and early HCC, which are difficult to discriminate by prior known biomarker techniques.

### DETAILED DESCRIPTION

In describing the present invention, the following terms will be employed, and are intended to be defined as indicated below.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The term “tumour” refers to refers to an abnormal mass of tissue resulting from a benign (non-cancerous) or malignant (cancerous) neoplastic process.

A “sample” as used herein may be a biological fluid, a cell or tissue sample (e.g. a biopsy), or an extract from which proteomic material can be obtained for proteome redox analysis. In particular, the sample may be a bodily fluid, such as a circulating bodily fluid. In particular, the sample may comprise a plasma sample, a blood sample, an exosome-containing circulating liquid sample, a cerebrospinal fluid sample, a lymphatic fluid sample, a ascites sample or pleural effusion sample. The sample may be one which has been freshly obtained from a subject or may be one which has been processed and/or stored prior to making a determination (e.g. frozen, fixed or subjected to one or more purification, enrichment or extraction steps). Further, the sample may be transported and/or stored, and collection may take place at a location remote from the place where the sample analysis and/or data analysis occurs.

The term “cancer” refers a disease caused by an uncontrolled division of abnormal cells in a part of the body.

The term “subject” or “patient” refers to all classes of animals, but in particular a mammal (e.g. a human, a non-human primate, a cat, dog, horse, donkey, sheep, pig, goat, cow, mouse, rat, rabbit or guinea pig), most particularly a human. The subject may have, or be suspected of having, a liver disease, including liver cirrhosis, hepatitis (e.g. hepatitis B or hepatitis C) and/or hepatocellular carcinoma.

The term “machine learning” refers to a type of artificial intelligence which can learn from provided datasets.

The term “predicting” may refer to determining at least one numerical or categorical value indicative of the presence of a disease or disease state, such as early-stage HCC.

The systems and methods described herein may be implemented in a computer system, in addition to the structural components and user interactions described. As used herein, the term “computer system” includes the hardware, software and data storage devices for embodying a system or carrying out a method according to the above-described embodiments. For example, a computer system may comprise a processing unit such as a central processing unit (CPU) and/or graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The following is presented by way of example and is not to be construed as a limitation to the scope of the claims.

### EXAMPLES

### Example 1: Materials and Methods

### 1.1 Patients & Plasma Samples

Plasma samples were obtained from healthy donors and patients with liver cirrhosis or early-stage HCC from the Royal Infirmary of Edinburgh.

### 1.2 Reagents

The materials used in the following examples include ultra pure 1M Tris-HCL pH 8.0, Invitrogen cat #15568-025G; Iodoacetamide (IAA light), Sigma-Aldrich cat #11149; Iodoacetamide-13C2, 2d2 (IAA heavy), Sigma-Aldrich cat #721328-0.25G; Sodium deoxycholate (SDC), Sigma-Aldrich cat #D6750-10G; DL-Dithiothreitol (DTT), Sigma-Aldrich cat #43819-5g; Trifluoroacetic acid (TFA), Sigma-Aldrich cat #T6508; Water LC-MS Grade, Sigma-Aldrich cat #1.15333; SpeadBead Magnetic Carboxylate (SP3 beads), Cytiva Cat #45152105050250 and cat #65152105050250; Acetonitrile (ACN), VWR cat #83639.320; ethanol absolute >99.8%, VWR cat #20821.365; Sequencing Grade Modified Trypsin, Promega cat #V511C; and AssayMAP Cartridge Rack, Reversed Phase S 5 μl, Agilent Technologies #G5496-60033.

Table 1 lists the buffers used for processing and labelling of peptides from plasma samples.

TABLE 1
Composition of buffers used for processing and labelling of
reduced and reversibly oxidised cysteine residues in proteins
from plasma samples.

| | | Final | Final |
|---|---|---|---|
| | | Concentration | Concentration |
| | Components in | of Component | in |
| Buffer | Stock solutions | in Buffer Mix | Sample |
| Alkylation | 1M Tris pH 8.0 | 0.1M | 0.1M |
| Mix 1 | | | |
| | 5% SDC | 2% | 2% |
| | 0.2M Alkylation 1 | 0.02M | 0.02M |
| | (IAA light) | | |
| | H₂O | | |
| DTT Mix | 1M Tris pH 8.0 | 0.1M | 0.1M |
| | 1M DTT | 0.1M | 0.033M |
| | H₂O | | |
| Alkylation | 1M Tris pH 8.0 | 0.1M | 0.1M |
| Mix 2 | | | |
| | 0.55M Alkylation | 0.1M | 0.04M |
| | 2 (IAA heavy) | | |
| | H2O | | |
| Trypsin Mix | 50 mM Acetic Acid | | |
| | Trypsin, 0.5 mg/ml | 0.1 mg/ml | 0.02 mg/ml |
| Priming | ACN | 50% | |
| Buffer | | | |
| | TFA | 0.1% | |
| Elution | ACN | 70% | |
| Buffer | | | |
| | TFA | 0.1% | |
| Equilibration | TFA | 0.1% | |
| and Cartridge | | | |
| Wash Buffer | | | |
| Digestion | 1M Tris pH 8.0 | 0.1M | |
| buffer | | | |
| A* | ACN | 5% | |
| | TFA | 0.1% | |

### 1.3 Sample Preparation and Labelling

Preparation and labelling of reduced and reversibly oxidised cysteine residues in peptides from plasma samples was performed using the AssayMAP Bravo Protein Sample Prep Platform (Agilent), using the protocol set out in the following steps.

Step 1: Samples and buffer are distributed in 96-well plates using the AssayMap BRAVO applications and utilities. The first labelling step was performed using 9 μl of Alkylation Mix 1 (Table 1), and 1 μl of plasma. Alkylation Mix 1 comprises SDC to solubilise plasma proteins, and IAA-light to label the free reduced thiol groups. Sample plate is sealed. Samples are boiled at 95° C. on a thermomixer at 1500 rpm for 5 min in the dark, and then incubated at 25° C. on a thermomixer at 1500 rpm for 1 hour.

In between each step the sample plate is sealed and taken off the Bravo platform and placed in a thermomixer C (Eppendorf) with a lid to protect the samples from evaporation and exposure to light. All plates are centrifuged before being put on the BRAVO platform to avoid air bubbles.

Step 2: From this step onwards the head is changed on the BRAVO Platform to the 96LT head and the SP3 program is used until the peptides are retrieved from the SP3 magnetic beads in step 5. 5 μl of DTT Mix (Table 1) is added to each sample to reduce reversibly oxidised cysteine residues. Sample plate is sealed and were then incubated at 25° C. in a thermomixer at 1500 rpm for 30 minutes in the dark.

Step 3: Reversibly oxidised cysteine residues were then labelled with IAA-heavy using Alkylation Mix 2 (Table 1). 10 μl Alkylation Mix 2 was added to the samples from Step 2. Samples were then incubated at 25° C. in a thermomixer at 1500 rpm for 1 hour in the dark.

Step 4:5 μl of washed SP3 beads are added to each sample followed by addition of 30 μl of 100% ACN. The dual-labelled proteins bind to the SP3 beads. After mixing, the SP3 beads are washed using 3×80% Ethanol washes to remove solvent from the previous steps, and prepare the proteins for digestion. Proteins are then digested using 65 μl digestion buffer and 5 μl of Trypsin Mix (Table 1). Samples are incubated at 37° C. on a thermomixer at 1500 rpm overnight using a heated lid.

Step 5: To acidify the peptides, 5 μl of 5% TFA is added to each well. Peptides are recovered from the magnetic beads by transferring the 70 μl sample to a well in a new plate. This step was repeated to prevent carry-over of magnetic beads that can compromise the Mass spectrometer.

Step 6: The head of the BRAVO is changed to the AssayMAP head and application peptide clean-up program was then run to desalt 30 μl of peptides using AssayMap Cartridges following the instruction in the application. Peptides are dried-down to 1-2 μl using a SpeedVac, and resuspended in 20 μl buffer A*.

### 1.4 UHPLC-Mass Spectrometry

For SICyLIA plasma proteomic experiments, tryptic digests are separated by nanoscale C18 reverse-phase liquid chromatography using an EASY-nLC II 1200 (Thermo Scientific) coupled to a Q-Exactive HF mass spectrometer (Thermo Scientific). Elution was carried out using a binary gradient with buffer A (2% acetonitrile) and B (80% acetonitrile), both containing 0.1% formic acid. Samples were loaded with 8 μl of buffer A into a 20 cm fused silica emitter (New Objective) packed in-house with ReproSil-Pur C18-AQ, 1.9 μm resin (Dr Maisch GmbH). Packed emitter was kept at 35° C. by means of a column oven (Sonation) integrated into the nanoelectrospray ion source (Thermo Scientific). Peptides were eluted at a flow rate of 300 ml/min using a binary gradient start at 3% of buffer B, kept at same percentage for 1 minutes, then increased to 23% over 42 minutes and then to 38% over 14 minutes. Finally, a column wash was performed increasing the % B to 95 and keeping it for 8 minutes, followed by a 5 minutes re-equilibration at 3% B for a total duration of 129 minutes. The eluting peptide solutions were automatically (online) electrosprayed into the mass spectrometer via a nanoelectrospray ion source (Sonation). An Active Background Ion Reduction Device (ABIRD) was used to decrease ambient contaminant signal level.

Mass spectrometry ionisation conditions used include spray voltage 2.1 kV, ion transfer tube temperature 250° C. Data were acquired using Xcalibur software (Thermo Scientific) and acquisition was carried out in positive ion mode using data independent acquisition (DIA), or data dependent acquisition. A full scan (FT-MS) over mass range of 340-1050 m/z was acquired at 60,000 resolution at 200 m/z, with a target value of 3,000,000 ions for a maximum injection time of 54 ms. Higher energy collisional dissociation fragmentation spectra were recorded at 30000 resolution at 200 m/z. All precursors were fragmented using 28 consecutive windows with 25 Da width, allowing for a 0.5 m/z overlap, covering a mass range from 350 to 1023 m/z. All ions were fragmented using normalised collision energy of 28, for a maximum injection time of 54 ms or a target value of 30,000 ions.

### 1.5 Proteomics Data Analysis

The MS Raw data were processed with Spectronaut version 17.3 using directDIA analysis querying UniProt Homo sapiens and MaxQuant contaminant database. The minimum peptide length was set to seven amino acids and specificity for trypsin/P cleavage was required, allowing up to two missed cleavage sites. Methionine oxidation and N-terminal acetylation were specified as variable modifications, as well as modification by light and heavy iodoacetamide on cysteine residues (carbamidomethylation). Compositions set in the software for carbamidomethylation heavy and light were: HNOCx(2)Hx(2) and H(3)NOC(2) respectively.

Minor peptide grouping was set to “Modified Sequence”, and Major and Minor Group Quantity were set to “Sum peptide quantity” and “Median precursor quantity” respectively. Single hit proteins were excluded from dataset, and all other parameters in Spectronaut were left to default values.

Spectronaut results for protein and peptide were exported and further analysed using Perseus software version 1.6.14.0. Intensity of cysteine containing peptides was normalised using intensity of the parent protein. Matrix was then filtered for 70% of valid values, and missing values were imputed separately for each column (width 0.3, down shift 1.8). Significantly regulated proteins between the three groups (healthy, cirrhotic and tumour) were selected using a permutation-based ANOVA test with FDR set at 1%.

MaxQuant output was further processed and analysed using Perseus software version 1.5.5.327. Peptides with Cys count lower than one were excluded, together with Reverse and Potential Contaminant flagged peptides. Protein level quantitation was done using the ProteinGroups.txt file. From the ProteinGroups.txt file, Reverse and Potential Contaminant flagged proteins were removed, and at least one uniquely assigned peptide and a minimum ratio count of 2 were required for a protein to be quantified. Only cysteine-containing peptides uniquely assigned to one protein group were normalised and included in the analysis.

### Example 2: SICyLIA Methods

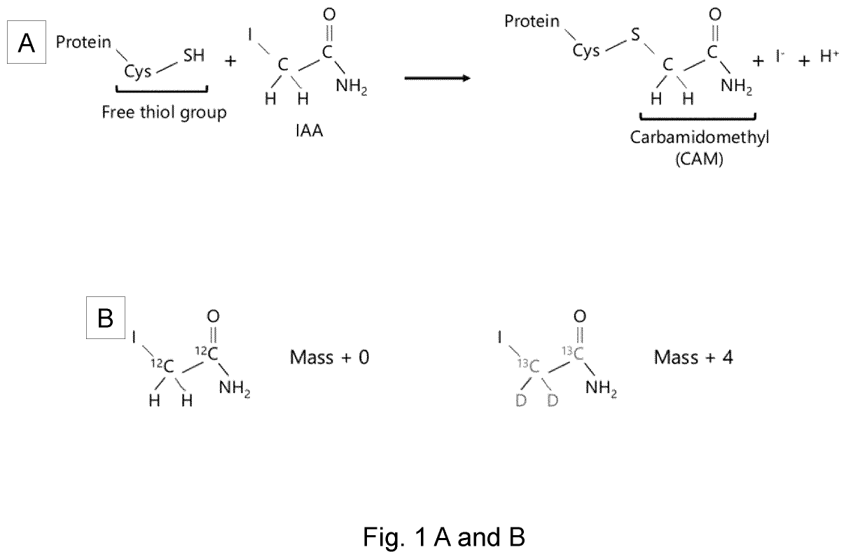

SICyLIA methods use IAA to label free reduced cysteine residues (FIG. 1A). IAA comprising light or heavy stable isotope-labelled carbon atoms (¹²C and H and ¹³C and Deuterium, respectively; FIG. 1B) may be used to label different types of cysteine residues, as described herein. IAA binds free reduced thiol groups, and does not bind residues with oxidative modifications, such as e.g. disulfide, glutathionylated, or nitrosylated. Upon binding to a free thiol group, IAA is converted into a carbamidomethyl (CAM) group.

Other moieties may be used to label cysteine residues, either instead of or in addition to IAA. For example, N-ethylmaleimide (NEM) is an example of another alkylating agent that reacts with free reduced thiol groups. NEM-light and NEM-heavy (containing ¹³C) could be used to distinguish different types of cysteine residues, or a combination of NEM-light and IAA-light.

### 2.1 SICyLIA Protocol for Detecting Reduced Cysteine Residues

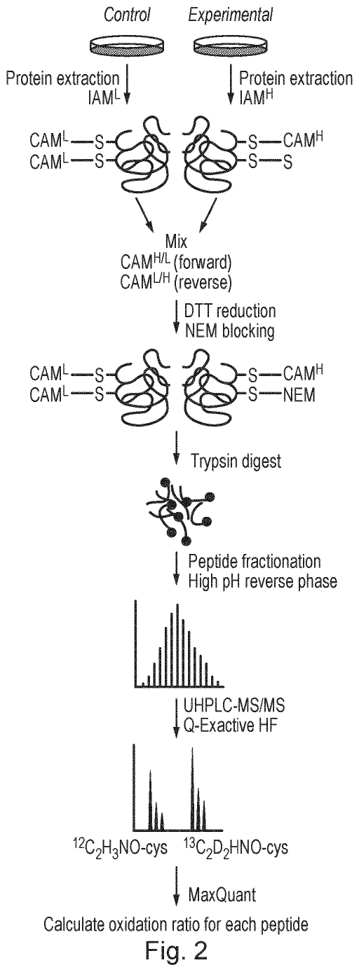

SICyLIA may be used to detect the relative levels of free reduced thiol groups in control samples compared to that of an experimental sample (FIG. 2). In the exemplified method peptides extracted from the control sample are labelled with IAA-light, and peptides from the experimental sample are labelled with IAA-heavy. The labels may be used in the alternative configuration (i.e. IAA-heavy labelling of control peptides and IAA-light labelling the experimental peptides; referred to in FIG. 2 as forward or reverse labelling). The samples are then mixed, and the proceeding steps performed simultaneously. The mixed sample is treated with DTT to reduce reversibly oxidised cysteine residues, and treated with NEM to block these groups. Peptides are then mixed together, digested with trypsin, and fractionated using HPLC before being subject to MS. The MS results are analysed computationally using the MaxQuant software, which provides a readout to calculate the oxidation ratio between the control and experimental sample.

Labelling of reduced thiol groups in two non-identical plasma samples may be performed using the AssayMAP Bravo platform, using a protocol similar to that described in section 1.3, with the labelling steps adjusted accordingly.

### 2.2 SICyLIA Protocol for Detecting Reduced and Reversibly Oxidised Cysteine Residues in the Same Sample

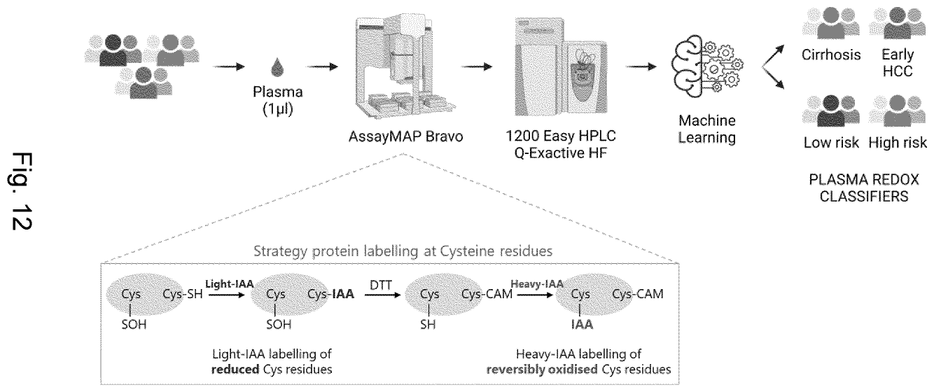

IAA-light and IAA-heavy may alternatively be used to differentiate between reduced free thiol groups and reversibly oxidised cysteine residues, to measure the abundance of these species in the same sample. In this protocol, incubation with DTT is required to reduce reversibly oxidised cysteine residues, which then enables them to be labelled with IAA-heavy (FIG. 12). This labelling method is employed in the protocol described in section 1.3.

### 2.3 SICyLIA Protocol for Plasma Samples

### 2.3.1 Methodology

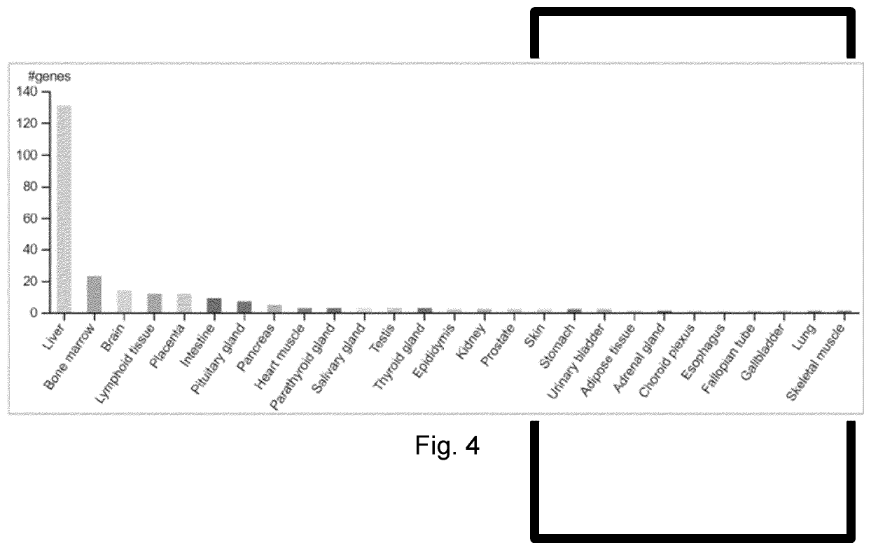

The inventors developed a method to apply the SICyLIA techniques to plasma samples (FIG. 12). A particular challenge with plasma samples is that it is difficult to measure the redox state of cysteine residues due to the highly oxidising environment of the plasma compared to inside of cells. Advantageously, the method of the present invention provides high sensitivity, and is able to detect many of the tissue-specific proteins present in the plasma (FIG. 4).

During Step 1, the samples undergo a first labelling step, preferably using with IAA-light. The present inventors discovered that subjecting the sample to elevated temperature (e.g. a boiling step-see 2.3.2 below) enhances labelling, but when doing so, there is a risk of deuterium loss if IAA-heavy is used as the first label.

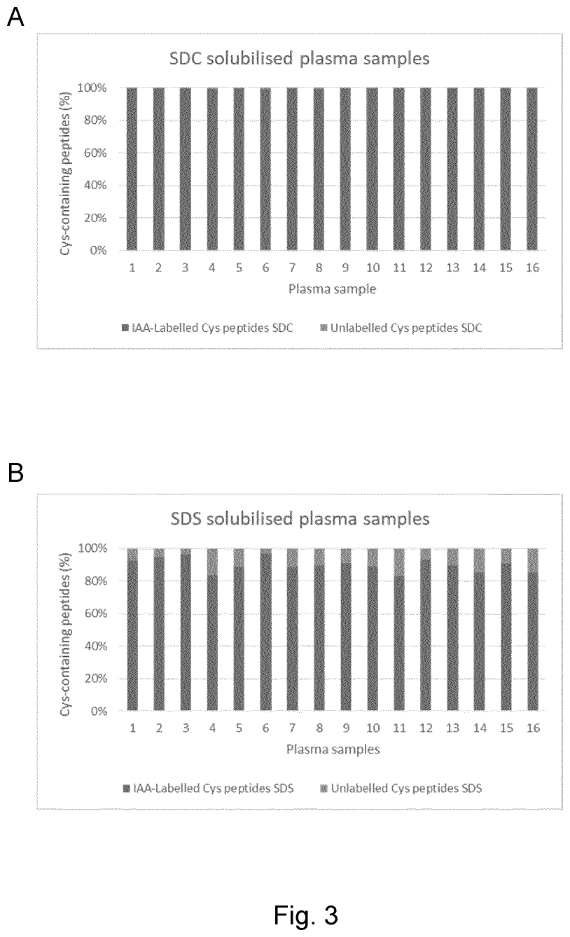

That is to say, IAA-light is advantageously employed as the first label, especially when an elevated temperature step is employed. Solubilisation of the plasma proteins was achieved using SDC, as the inventors found that when instead using SDS, a subset of cysteine residues were not labelled. This is demonstrated in FIG. 3, wherein the percentage of unlabelled cysteine-containing peptides is clearly visible at around 5-15% for SDS-solubilised plasma samples (FIG. 3B) whereas virtually all cysteine-containing peptide were IAA-labelled following SDC-solubilisation (FIG. 3A). Samples may optionally be boiled during Step 1 (see 2.3.2 below), which increases the labelling efficiency.

In Step 2, the reversibly oxidised cysteine residues in the samples are reduced with DTT. In Step 3, these same residues are then labelled with the IAA isotope that was not used in step 1. When an elevated temperature is employed in step 1, it is advantageous to use IAA-light as the first label and then use IAA-heavy as the second label in step 2. For example, the free reduced residues may be labelled with IAA-light, and the reversibly oxidised residues may be labelled with IAA-heavy. Importantly, because IAA-light is not removed after step 1, the concentration of DTT should preferably be sufficiently greater than that of IAA-light to ensure that any excess IAA-light is quenched by DTT and cannot bind the reversibly oxidised residues. The concentration of IAA-heavy should preferably also be sufficiently greater than IAA-light to ensure that i) there is enough for labelling all of the previously reversibly oxidised residues, which are more abundant than reduced residues in plasma, and ii) there is enough such that reduction of IAA-heavy by excess DTT is inconsequential (i.e. IAA-heavy is not quenched by DTT). The inventors found that using 0.02 M label for reduced residues, 0.033 M DTT, and 0.04 M label for reversibly oxidised residues enabled effective labelling of both redox states in the same sample.

In Step 4, the proteins are digested with trypsin to generate peptides that are suitable for downstream analysis using UHPLC-MS/MS.

In Step 5, peptides are recovered and purified.

In step 6, peptides containing cysteine residue labels (IAA-light and IAA-heavy) are fractionated by UHPLC connected online to the MS and analysed by MS.

### 2.3.2 Investigating the Effects of Boiling Plasma Samples During Sample Preparation

To investigate strategies for increasing the sensitivity of SICyLIA in plasma samples (FIG. 4), the present inventors tested the effect of boiling plasma samples before subjecting them to SICyLIA. Human plasma samples in triplicate were boiled, and non-boiled samples were used as a control. SICyLIA labelling was performed as described in section 1.3, using IAA-light and NEM-light to label reduced and reversibly oxidised cysteine residues, respectively.

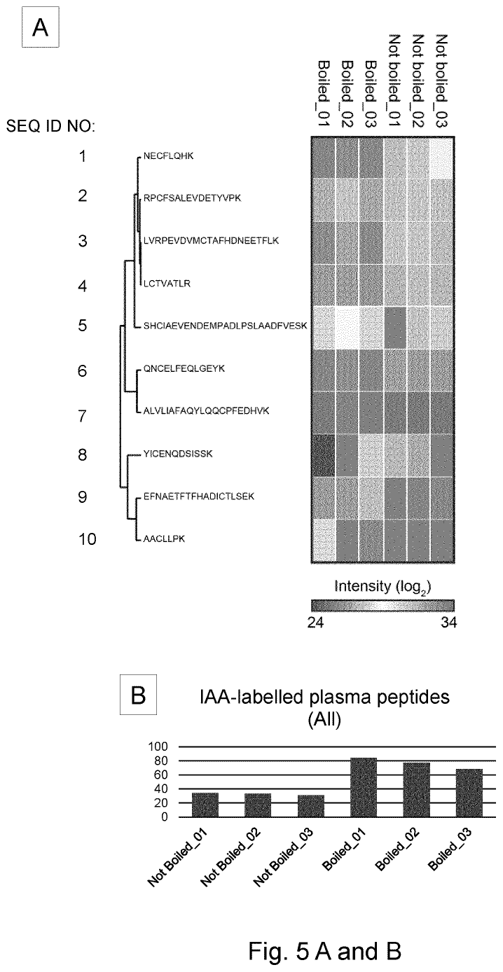

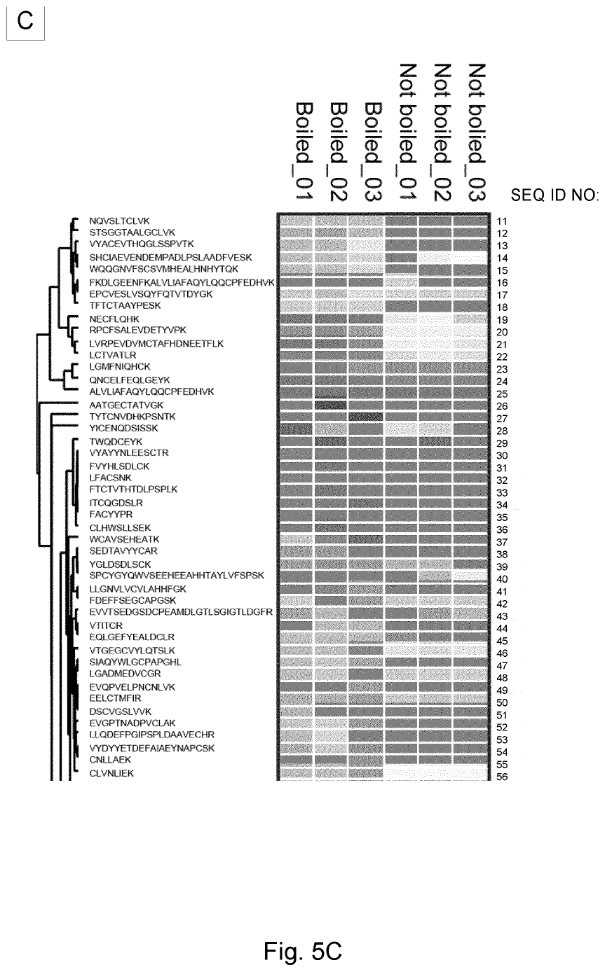

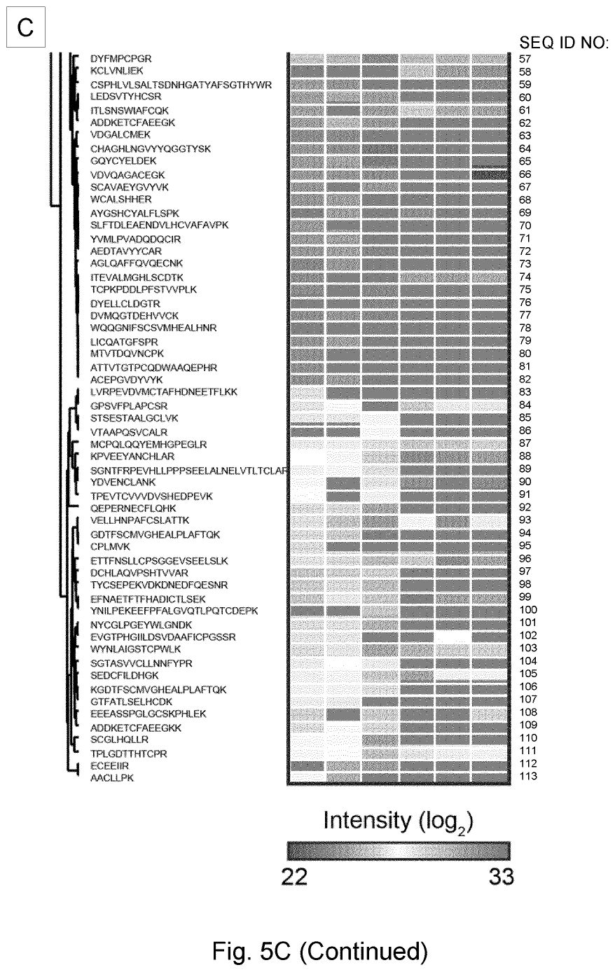

The results are shown in FIG. 5. FIG. 5A shows the intensity level of 10 different albumin peptides comprising reduced cysteine residues, labelled with IAA-light and identified by MS. Boiling the samples resulted in the detection of increased levels for 6 of the peptides identified, and one peptide (shown at the bottom of FIG. 5A) was only detectable in the boiled samples. Quantification of the number of peptides in the boiled and non-boiled samples using the MaxQuant software confirmed that boiling results in a significant increase in the number of peptides found labelled with IAA on cysteine residues (FIG. 5B).

The inventors also investigated the presence of a broader range of peptides in the boiled and non-boiled samples, deriving from different proteins (FIG. 5C). The results confirmed that boiling the samples greatly increased the efficiency of labelling, as many more IAA-labelled peptides were identified that were not identifiable in non-boiled samples, even when the concentration of IAA-light (as used in this experiment) was relatively low.

### 2.3.3 Investigating the Efficacy of Sample Processing on Beads

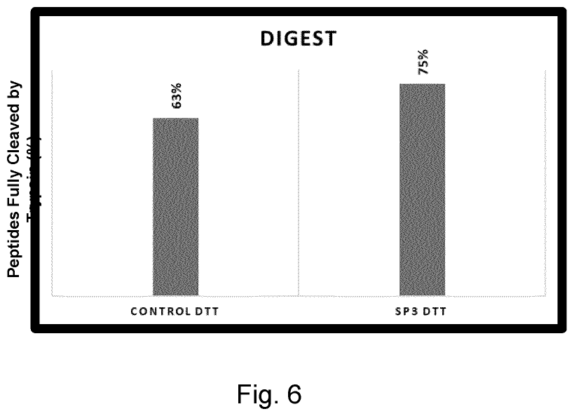

The efficacy of trypsin digestion of IAA-labelled peptides was assessed in control conditions, and using SP3 magnetic beads. Using SP3 beads resulted in an increased digest efficiency (FIG. 6). Without wishing to be bound by any particular theory, the present inventors believe the beads enable easier washing of the proteins, more efficiently removing buffer components that negatively impact trypsin function, for example, DTT, IAA, and precipitated SPC.

### Example 3: Use of SICyLIA for Detecting Changes in Redox State in Hepatocellular Carcinoma (HCC) Plasma Samples

### 3.1 Experimental Setup and Patient Overview

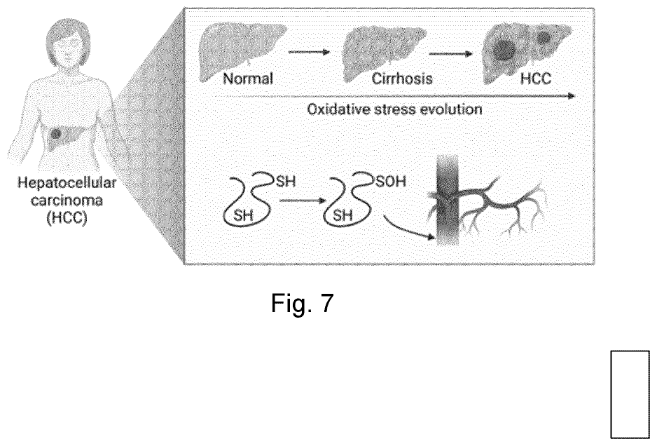

SICyLIA was performed on plasma samples to determine whether redox changes in plasma proteins could be used to predict the likelihood of disease and/or to discriminate between disease states (e.g. hard to discriminate adjacent disease states such as liver cirrhosis and early-stage HCC). As an example, the inventors applied the technology to assess patients with liver cirrhosis and early-stage HCC. The inventors hypothesised that SICyLIA could be used to identify changes in levels of cysteine oxidation present in disease states (FIG. 7). Notably, the effect of oxidative stress on specific cysteine residues can be complex. Conformational changes in certain proteins may expose or conceal cysteine residues such that thiol groups are rendered more or less susceptible to oxidation. Proteins differ in the degree to which they are influenced by oxidative stress as, for example, redox enzymes may be directly involved in the body's response to an oxidative insult and may be actively reduced and/or cycled as part of that response. Moreover, some cysteine residues may be subject to irreversible oxidation, which means those residues will not be labelled even after treatment with a reducing agent such as DTT (i.e. the irreversibly oxidised cysteines will be absent from the measure of reduced and reversibly oxidised cysteine residues). Therefore, higher levels of oxidative stress can, counterintuitively, manifest as decreased levels of both reduced cysteines and reversibly oxidised cysteines. This complex plasma proteome redox signal therefore has the potential to convey considerable disease-state specific information.

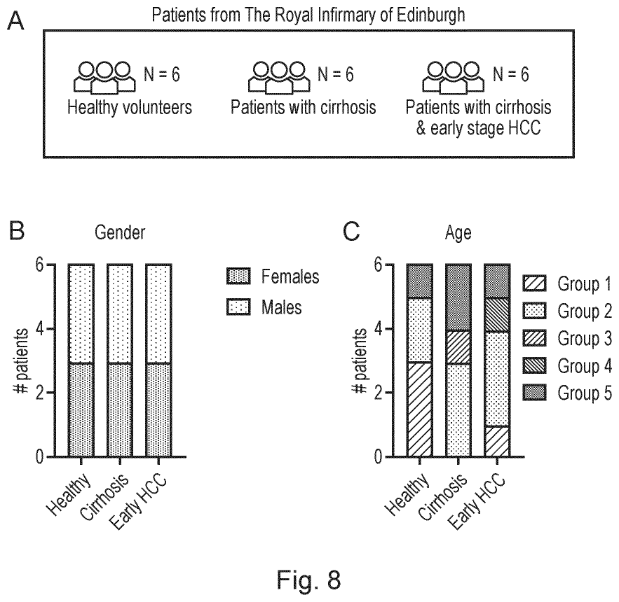

To study the implications further, plasma samples were obtained from 6 healthy volunteers as a control group, 6 patients with cirrhosis, and 6 patients with cirrhosis and early-stage HCC (FIG. 8A). Each group consisted of 3 females and 3 males (FIG. 8B), and the patients in each of the groups had a similar profile of ages (FIG. 8C).

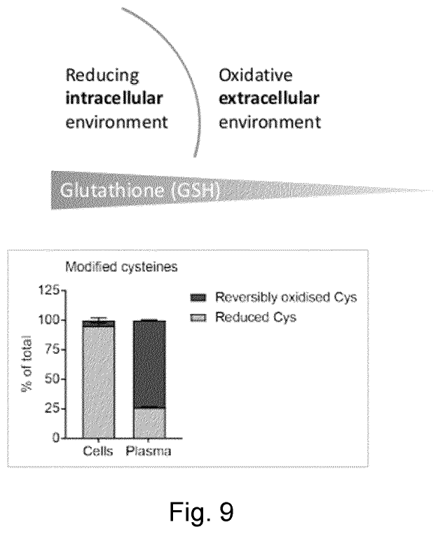

Plasma samples obtained from the patients were subjected to SICyLIA using a dual-labelling approach, wherein reduced cysteine residues were labelled with IAA-light, and reversibly oxidised cysteine residues were labelled with NEM-light. Using dual labelling enables a greater amount of information to be derived from the plasma samples, as the oxidative environment means that there is a much higher proportion of reversibly oxidised cysteine residues compared to that found inside cells (FIG. 9). The labelled samples were analysed using UHPLC-MS/MS, as described herein, and the results were analysed using MaxQuant and Perseus software.

### 3.2 Proteomics Analysis

Proteomics analysis was performed on the 18 patient samples, to identify whether redox changes could be used to distinguish between healthy donors, patients with cirrhosis, and patients with early HCC. Discriminating between the latter two patient groups is a particularly important goal, because prior known biomarker-based methods to distinguish between liver cirrhosis and early-stage HCC exhibit relatively poor performance.

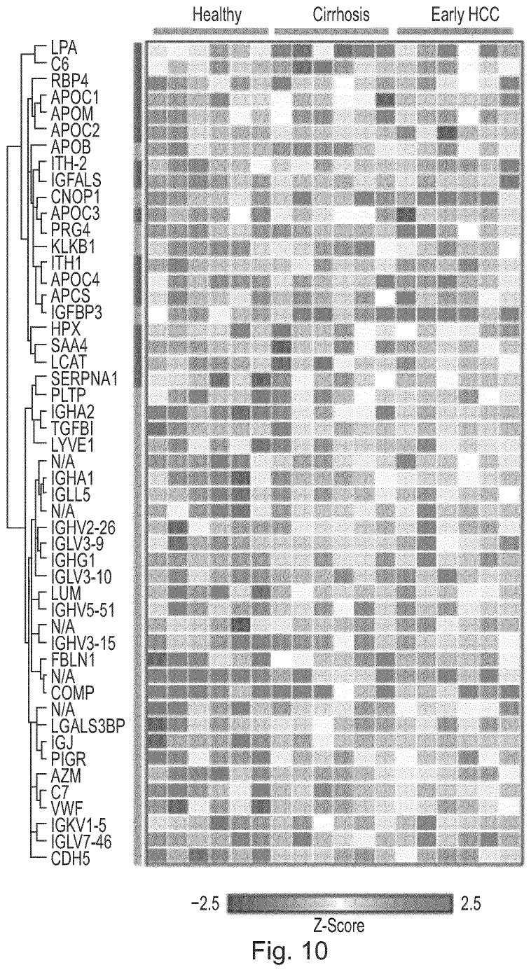

The overall protein levels in the plasma samples were determined by the MaxQuant software using non-labelled peptides. FIG. 10 shows proteins where the plasma level was determined to be significantly different between the patient subgroups, using an ANOVA test with a 5% false discovery rate. These results demonstrate that a number of proteins are present at different levels in the plasma of healthy donors, compared to that of plasma from patients with cirrhosis or early HCC. However, the differences in protein levels between cirrhosis and early-stage HCC are less apparent. This underscores the challenge to provide a biomarker-based method to discriminate between liver cirrhosis patients (who do not have HCC) and those patients who have early-stage (possibly treatable) HCC. Traditional proteomics-based signatures that rely on protein abundance may be poorly suited to this challenge.

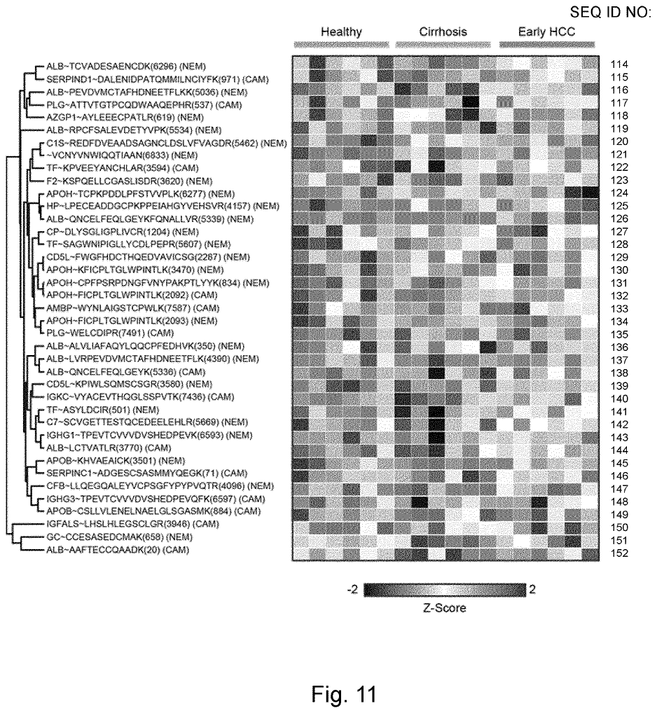

The inventors then quantified the redox changes observed for certain proteins, in healthy donors and patients with cirrhosis or early HCC. FIG. 11 shows that many peptides have differential redox changes in certain patient groups. For example, many peptides from ALB show increased amounts of reduced/oxidised cysteine residues in patients with early HCC, and different changes change in patients with cirrhosis. That is to say, the results shown in FIG. 11 support the use of the plasma proteome cysteine redox signal as a biomarker to distinguish not only healthy subjects from disease state, but also to discriminate between the disease states of liver cirrhosis and early-stage HCC. Therefore, utilising the SICyLIA method, adapted as described herein to plasma samples, provides a superior approach to generating a signature—and related machine learning classifier—as compared with traditional proteomics approaches that rely on protein abundance rather than redox status. Without wishing to be bound by any particular theory, the present inventors believe that using cysteine oxidation levels (alone or in combination with protein levels) will provide better-performing models for detection of early cancer.

### 3.1 SICyLIA-based Screening Platform for the Detection of HCC

The results shown in FIG. 11 demonstrates that redox changes measured by SICyLIA in plasma samples could be used to generate a classifier that identifies the likelihood a patient suffers from cirrhosis or early-stage HCC. FIG. 12 exemplifies a pipeline that may be employed for such analyses. Plasma samples as small as 1 μl (or even less) in volume may be taken, for example using a pin prick blood test method, and subject to SICyLIA. The labelling may comprise single or dual labelling of either reduced cysteines, or both reduced and reversibly oxidised cysteines. Advantageously, a dual labelling method is used as described in section 2.2, which provides a greater amount of information regarding the redox status of residues in each sample. The labelled samples are then subject to UHPLC-MS/MS, and the data is analysed using software such as MaxQuant or Spectronaut. A machine learning approach may then be applied to classify subjects into different patient groups based on the redox status of certain cysteine residues. For example, peptides from proteins including e.g. ALB, APOH, IGHG1, and SERPINC1 show differential changes in redox status between the 3 patient subgroups, and the redox state of such peptides could therefore be used as biomarkers in a classifier.

Table 2 shows the protein, cysteine-containing peptide fragment and SEQ ID No. for each of the peptides of FIG. 11.

TABLE 2
Details of peptides of FIG. 11.

| Protein | UniProt ID | | | |
|---|---|---|---|---|
| (Gene | (release | | | |
| Name) | 2023_01) | Sequence | Label | SEQ ID NO: |
| ALB | P02768 | TCVADESAENCDK | NEM | 114 |
| SERPIND1 | P05546 | DALENIDPATQMMILNCIYFK | CAM | 115 |
| ALB | P02768 | PEVDVMCTAFHDNEETFLKK | NEM | 116 |
| PLG | P00747 | ATTVTGTPCQDWAAQEPHR | CAM | 117 |
| AZGP1 | P25311 | AYLEEECPATLR | NEM | 118 |
| ALB | P02768 | RPCFSALEVDETYVPK | NEM | 119 |
| C1S | P09871 | REDEDVEAADSAGNCLDSLVEVAGDR | NEM | 120 |
| — | — | VCNYVNWIQQTIAAN | NEM | 121 |
| TF | P02787 | KPVEEYANCHLAR | CAM | 122 |
| F2 | P00734 | KSPQELLCGASLISDR | NEM | 123 |
| APOH | P02749 | TCPKPDDLPFSTVVPLK | NEM | 124 |
| HP | P00738 | LPECEADDGCPKPPEIAHGYVEHSVR | NEM | 125 |
| ALB | P02768 | QNCELFEQLGEYKFQNALLVR | NEM | 126 |
| CP | P00450 | DLYSGLIGPLIVCR | NEM | 127 |
| TF | P02787 | SAGWNIPIGLLYCDLPEPR | NEM | 128 |
| CD5L | O43866 | FWGFHDCTHQEDVAVICSG | NEM | 129 |
| APOH | P02749 | KFICPLTGLWPINTLK | NEM | 130 |
| APOH | P02749 | CPFPSRPDNGFVNYPAKPTLYYK | NEM | 131 |
| APOH | P02749 | FICPLTGLWPINTLK | CAM | 132 |
| AMBP | P02760 | WYNLAIGSTCPWLK | CAM | 133 |
| APOH | P02749 | FICPLTGLWPINTLK | NEM | 134 |
| PLG | P00747 | WELCDIPR | CAM | 135 |
| ALB | P02768 | ALVLIAFAQYLQQCPFEDHVK | NEM | 136 |
| ALE | P02768 | LVRPEVDVMCTAFHDNEETFLK | NEM | 137 |
| ALB | P02768 | QNCELFEQLGEYK | CAM | 138 |
| CD5L | O43866 | KPIWLSQMSCSGR | NEM | 139 |
| IGKC | P01834 | VYACEVTHQGLSSPVTK | CAM | 140 |
| TF | P02787 | ASYLDCIR | NEM | 141 |
| C7 | P10643 | SCVGETTESTQCEDEELEHLR | NEM | 142 |
| IGHG1 | P01857 | TPEVTCVVVDVSHEDPEVK | NEM | 143 |
| ALB | P02768 | LCTVATLR | CAM | 144 |
| APOB | P04114 | KHVAEAICK | NEM | 145 |
| SERPINC1 | P01008 | ADGESCSASMMYQEGK | CAM | 146 |
| CFB | P00751 | LLQEGQALEYVCPSGFYPYPVQTR | NEM | 147 |
| IGHG3 | P01860 | TPEVTCVVVDVSHEDPEVQFK | CAM | 148 |
| APOB | P04114 | CSLLVLENELNAELGLSGASMK | CAM | 149 |
| IGFALS | P35858 | LHSLHLEGSCLGR | CAM | 150 |
| GC | P02774 | CCESASEDCMAK | NEM | 151 |
| ALB | P02768 | AAFTECCQAADK | CAM | 152 |

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A method for measuring proteomic cysteine redox status in a sample obtained from bodily fluid of a subject, the method comprising:
(i) providing said sample, wherein the sample comprises a plurality of different proteins that comprise cysteine residues, a proportion of which are in reduced form and a proportion of which are in reversibly oxidised form;
(ii) contacting the sample with a first label that selectively labels the reduced cysteine thiols of the plurality of different proteins;
(iii) contacting the sample with a reducing agent that reduces the reversibly oxidised cysteine residues to form reduced cysteine thiols;
(iv) contacting the sample with a second label that labels the reduced cysteine thiols formed in step (iii), wherein the second label is distinguishable from the first label by mass spectrometry,
(v) processing the sample for analysis by mass spectrometry; and
(vi) analysing the sample by mass spectrometry to measure at least the relative abundance of identified peptides labelled with the first label and corresponding identified peptides labelled with the second label, thereby providing a measure of the proteomic cysteine redox status of the sample.

2. The method of claim 1, wherein said sample has been obtained from a circulating bodily fluid.

3. The method of claim 2, wherein the circulating bodily fluid is selected from: a plasma sample, a blood sample, a circulating exosome-containing liquid sample, a cerebrospinal fluid sample and a lymphatic fluid sample.

4. The method of claim 1, wherein said sample has been obtained from ascites or pleural effusion.

5. The method of any one of the preceding claims, wherein said sample is heated to a temperature of greater than 50° C., optionally greater than 90° C., for a period of time sufficient to increase protein denaturation prior to or current with contacting the sample with said first label.

6. The method of any one of the preceding claims, wherein said first label is selected from: iodoacetamide-light (“IAA-light”), n-ethylmaleimide (NEM), and heavy stable isotope-labelled iodoacetamide (“IAA-heavy”).

7. The method of any one of the preceding claims, wherein said second label is selected from: IAA-heavy, IAA-light and n-ethylmaleimide (NEM), provided that said second label differs from said first label.

8. The method of any one of the preceding claims, wherein said first label comprises IAA-light and said second label comprises IAA-heavy.

9. The method of any one of the preceding claims, wherein said reducing agent comprises dithiothreitol (DTT).

10. The method of any one of the preceding claims, wherein said sample is solubilised by contacting with sodium deoxycholate (SDC) prior to or during step (ii).

11. The method of any one of the preceding claims, wherein said sample is contacted with magnetic beads, optionally SpeedBead Magnetic Carboxylate (SP3 beads), prior to or during step (v).

12. The method of any one of the preceding claims, wherein the method further comprises contacting the sample with at least one tandem mass tag (TMT) to enable multiplexing of said mass spectrometry analysis.

13. The method of any one of the preceding claims, wherein the concentration of said second label in step (iv) is higher than the concentration of said first label in step (ii).

14. The method of claim 13, wherein the first label comprises IAA-light at a concentration during labelling of between 0.015M and 0.025M, optionally at 0.02M, and wherein the second label comprises IAA-heavy at a concentration during labelling of between 0.03M and 0.05M, optionally at 0.04M.

15. The method of claim 14, wherein the reducing agent comprises DTT at a concentration during the reducing step of between 0.025M and 0.045M, optionally at 0.033M.

16. The method of any one of the preceding claims, wherein said processing of step (v) comprises at least one of: enzymatic digestion of the proteins in the sample, acidification of the sample, and peptide fractionation of the sample.

17. The method of claim 16, wherein said enzymatic digestion is trypsin digestion.

18. The method of any one of the preceding claims, wherein the mass spectrometry (MS) comprises ultra-high performance liquid chromatography-MS/MS (UHPLC-MS/MS).

19. The method of any one of the preceding claims, wherein step (vi) further comprises calculating, for each of a plurality of cysteine residues, the proportion that was in reversibly oxidised form in the sample.

20. The method of any one of the preceding claims, wherein step (vi) further comprises deriving at least one statistical measure, optionally a z-score, for the degree of cysteine oxidation of each of a plurality of non-identical peptides.

21. The method of any one of the preceding claims, wherein said plurality of different proteins comprises at least 50, 100, 500, or at least 1000.

22. The method of any one of the preceding claims, wherein said sample is of less than 100 μl, less than 50 μl, less than 10 μl, less than 1 μl or less than 100 nl when obtained from the subject.

23. The method of any one of the preceding claims, wherein the method further comprises normalising the measure of identified peptides labelled with the first label and/or the measure of identified peptides labelled with the second label for protein abundance.

24. The method of claim 23, wherein said normalising comprises normalising for the level of the corresponding cysteine-containing protein in the sample as determined by mass spectrometry.

25. A method for identifying biomarkers that are significantly associated with a particular disease state, for identifying biomarkers that are predictive of response to a treatment and/or for identifying biomarkers for monitoring disease progression, the method comprising:
performing the method of any one of claims 1 to 24 on a plurality of samples obtained from subjects known not to have the particular disease state (“controls”) and on a plurality of samples obtained from subjects known to have the particular disease state (“diseased subjects”) and thereby determining the proteomic cysteine redox status of the controls and the diseased subjects for a plurality of proteins and/or peptides;
comparing the proteomic cysteine redox status of the controls and the diseased subjects to identify proteins and/or peptides that are significantly oxidised or reduced in the samples from the diseased subjects as compared with the samples from the controls, thereby identifying these proteins and/or peptides as biomarkers of the particular disease state.

26. The method of claim 25, wherein the subjects known not to have the particular disease state have a different disease or have the same disease as the particular disease state, but are at a different disease stage.

27. The method of claim 25, wherein the subjects known not to have the particular disease state are healthy controls.

28. The method of claim 25, wherein the particular disease state is a liver disease and/or a cancer.

29. The method of claim 26, wherein the particular disease state is early-stage hepatocellular carcinoma (HCC).

30. The method of claim 29, wherein the subjects known not to have the particular disease state are subjects who are known to have liver cirrhosis without HCC; are subjects who are known to have hepatitis B without HCC; and/or are subjects who are known to have hepatitis C without HCC.

31. The method of claim 30, wherein the particular disease state is a metastatic cancer of known primary and the subjects known not to have the particular disease state have a cancer, optionally a metastatic cancer, of a primary source that differs from said known primary of the particular disease state.

32. A method for determining that a subject is likely to have hepatocellular carcinoma (HCC), optionally early-stage HCC, the method comprising:
performing the method of any one of claims 1 to 24 on a bodily fluid sample obtained from the subject thereby determining the proteomic cysteine redox status of the sample for a plurality of proteins and/or peptides;
providing data that represents the proteomic cysteine redox status of the sample for the plurality of proteins and/or peptides to a machine learning classifier, wherein said classifier has been trained on a training dataset that comprises proteomic cysteine redox status for the same plurality of proteins and/or peptides from both subjects known to have HCC and subjects known not to have HCC;
causing the machine learning classifier to classify the sample as belonging to the HCC class or not the HCC class based on the provided proteomic cysteine redox status of the sample; and
outputting the classification to a user.

33. The method of claim 32, wherein the subject has, or is suspected to have, liver cirrhosis.

34. The method of claim 32 or claim 33, wherein the machine learning classifier has been trained on a training data set that comprises proteomic cysteine redox status from subjects known to have early-stage HCC and subjects known to have liver cirrhosis without HCC.

35. The method of any one of claims 32 to 34, wherein the machine learning classifier comprises a processor and non-transitory memory comprising instructions that when run by the processor cause the processor to process the data representing the proteomic cysteine redox status of the sample for the plurality of proteins and/or peptides according to a learnt model and to output a probabilistic classification of HCC or non-HCC.

36. The method of claim 35, wherein the learnt model is selected from: a decision tree, a logistic regression model, an artificial neural network, a support vector machine (SVM), naïve bayes or k-nearest neighbour algorithm, optionally wherein the decision tree comprises a gradient boosting algorithm or a random forest algorithm.

37. The method of any one of claims 32 to 36, wherein said plurality of proteins and/or peptides comprises at least 5, 10, 15 or at least 20 proteins, or cysteine-containing peptide fragments thereof, selected from: GC, ALB, IGFALS, SERPIND1, AZGP1, PLG, IGKC, CD5L, TF, C7, IGHG1, CFB, IGHG3, APOB, SERPINC1, F2, C1S, CP, CD5L, APOH, AMBP, PLG, and HP.

38. The method of any one of claims 32 to 37, wherein said plurality of proteins and/or peptides comprises at least 5, 10, 15, 20 or all of the peptides set forth in SEQ ID NOs: 114-152.

39. The method of any one of claims 32 to 38, wherein the machine learning classifier is also provided with one or more additional biomarker measurements, optionally protein abundance measurements, obtained from the sample, and wherein the machine learning classifier has been trained on a training data set that further comprises the one or more additional biomarker measurements from both subjects known to have HCC and subjects known not to have HCC.

40. The method of claim 39, where the one or more additional biomarker measurements comprise: the blood level of alpha-fetoprotein (AFP) and/or any one of the proteins shown in FIG. 10.


SEQUENCE LISTING (152 sequences)

SEQ ID NO: 1 - protein, 8 aa, synthetic construct
NECFLQHK

SEQ ID NO: 2 - protein, 16 aa, synthetic construct
RPCFSALEVDETYVPK

SEQ ID NO: 3 - protein, 22 aa, synthetic construct
LVRPEVDVMCTAFHDNEETFLK

SEQ ID NO: 4 - protein, 8 aa, synthetic construct
LCTVATLR

SEQ ID NO: 5 - protein, 27 aa, synthetic construct
SHCIAEVENDEMPADLPSLAADFVESK

SEQ ID NO: 6 - protein, 13 aa, synthetic construct
QNCELFEQLGEYK

SEQ ID NO: 7 - protein, 21 aa, synthetic construct
ALVLIAFAQYLQQCPFEDHVK

SEQ ID NO: 8 - protein, 12 aa, synthetic construct
YICENQDSISSK

SEQ ID NO: 9 - protein, 19 aa, synthetic construct
EFNAETFTFHADICTLSEK

SEQ ID NO: 10 - protein, 7 aa, synthetic construct
AACLLPK

SEQ ID NO: 11 - protein, 10 aa, synthetic construct
NQVSLTCLVK

SEQ ID NO: 12 - protein, 14 aa, synthetic construct
STSGGTAALGCLVK

SEQ ID NO: 13 - protein, 17 aa, synthetic construct
VYACEVTHQGLSSPVTK

SEQ ID NO: 14 - protein, 27 aa, synthetic construct
SHCIAEVENDEMPADLPSLAADFVESK

SEQ ID NO: 15 - protein, 23 aa, synthetic construct
WQQGNVFSCSVMHEALHNHYTQK

SEQ ID NO: 16 - protein, 31 aa, synthetic construct
FKDLGEENFKALVLIAFAQYLQQCPFEDHVK

SEQ ID NO: 17 - protein, 20 aa, synthetic construct
EPCVESLVSQYFQTVTDYGK

SEQ ID NO: 18 - protein, 12 aa, synthetic construct
TFTCTAAYPESK

SEQ ID NO: 19 - protein, 8 aa, synthetic construct
NECFLQHK

SEQ ID NO: 20 - protein, 16 aa, synthetic construct
RPCFSALEVDETYVPK

SEQ ID NO: 21 - protein, 22 aa, synthetic construct
LVRPEVDVMCTAFHDNEETFLK

SEQ ID NO: 22 - protein, 8 aa, synthetic construct
LCTVATLR

SEQ ID NO: 23 - protein, 10 aa, synthetic construct
LGMFNIQHCK

SEQ ID NO: 24 - protein, 13 aa, synthetic construct
QNCELFEQLGEYK

SEQ ID NO: 25 - protein, 21 aa, synthetic construct
ALVLIAFAQYLQQCPFEDHVK

SEQ ID NO: 26 - protein, 12 aa, synthetic construct
AATGECTATVGK

SEQ ID NO: 27 - protein, 14 aa, synthetic construct
TYTCNVDHKPSNTK

SEQ ID NO: 28 - protein, 12 aa, synthetic construct
YICENQDSISSK

SEQ ID NO: 29 - protein, 8 aa, synthetic construct
TWQDCEYK

SEQ ID NO: 30 - protein, 13 aa, synthetic construct
VYAYYNLEESCTR

SEQ ID NO: 31 - protein, 10 aa, synthetic construct
FVYHLSDLCK

SEQ ID NO: 32 - protein, 7 aa, synthetic construct
LFACSNK

SEQ ID NO: 33 - protein, 15 aa, synthetic construct
FTCTVTHTDLPSPLK

SEQ ID NO: 34 - protein, 9 aa, synthetic construct
ITCQGDSLR

SEQ ID NO: 35 - protein, 7 aa, synthetic construct
FACYYPR

SEQ ID NO: 36 - protein, 10 aa, synthetic construct
CLHWSLLSEK

SEQ ID NO: 37 - protein, 11 aa, synthetic construct
WCAVSEHEATK

SEQ ID NO: 38 - protein, 11 aa, synthetic construct
SEDTAVYYCAR

SEQ ID NO: 39 - protein, 10 aa, synthetic construct
YGLDSDLSCK

SEQ ID NO: 40 - protein, 28 aa, synthetic construct
SPCYGYQWVSEEHEEAHHTAYLVFSPSK

SEQ ID NO: 41 - protein, 16 aa, synthetic construct
LLGNVLVCVLAHHFGK

SEQ ID NO: 42 - protein, 14 aa, synthetic construct
FDEFFSEGCAPGSK

SEQ ID NO: 43 - protein, 30 aa, synthetic construct
EVVTSEDGSDCPEAMDLGTLSGIGTLDGFR

SEQ ID NO: 44 - protein, 6 aa, synthetic construct
VTITCR

SEQ ID NO: 45 - protein, 14 aa, synthetic construct
EQLGEFYEALDCLR

SEQ ID NO: 46 - protein, 13 aa, synthetic construct
VTGEGCVYQTSLK

SEQ ID NO: 47 - protein, 15 aa, synthetic construct
SIAQYWLGCPAPGHL

SEQ ID NO: 48 - protein, 11 aa, synthetic construct
LGADMEDVCGR

SEQ ID NO: 49 - protein, 14 aa, synthetic construct
EVQPVELPNCNLVK

SEQ ID NO: 50 - protein, 9 aa, synthetic construct
EELCTMFIR

SEQ ID NO: 51 - protein, 10 aa, synthetic construct
DSCVGSLVVK

SEQ ID NO: 52 - protein, 14 aa, synthetic construct
EVGPTNADPVCLAK

SEQ ID NO: 53 - protein, 21 aa, synthetic construct
LLQDEFPGIPSPLDAAVECHR

SEQ ID NO: 54 - protein, 21 aa, synthetic construct
VYDYYETDEFAIAEYNAPCSK

SEQ ID NO: 55 - protein, 7 aa, synthetic construct
CNLLAEK

SEQ ID NO: 56 - protein, 8 aa, synthetic construct
CLVNLIEK

SEQ ID NO: 57 - protein, 9 aa, synthetic construct
DYFMPCPGR

SEQ ID NO: 58 - protein, 9 aa, synthetic construct
KCLVNLIEK

SEQ ID NO: 59 - protein, 28 aa, synthetic construct
CSPHLVLSALTSDNHGATYAFSGTHYWR

SEQ ID NO: 60 - protein, 11 aa, synthetic construct
LEDSVTYHCSR

SEQ ID NO: 61 - protein, 13 aa, synthetic construct
ITLSNSWIAFCQK

SEQ ID NO: 62 - protein, 13 aa, synthetic construct
ADDKETCFAEEGK

SEQ ID NO: 63 - protein, 9 aa, synthetic construct
VDGALCMEK

SEQ ID NO: 64 - protein, 18 aa, synthetic construct
CHAGHLNGVYYQGGTYSK

SEQ ID NO: 65 - protein, 10 aa, synthetic construct
GQYCYELDEK

SEQ ID NO: 66 - protein, 11 aa, synthetic construct
VDVQAGACEGK

SEQ ID NO: 67 - protein, 12 aa, synthetic construct
SCAVAEYGVYVK

SEQ ID NO: 68 - protein, 9 aa, synthetic construct
WCALSHHER

SEQ ID NO: 69 - protein, 14 aa, synthetic construct
AYGSHCYALFLSPK

SEQ ID NO: 70 - protein, 22 aa, synthetic construct
SLFTDLEAENDVLHCVAFAVPK

SEQ ID NO: 71 - protein, 14 aa, synthetic construct
YVMLPVADQDQCIR

SEQ ID NO: 72 - protein, 11 aa, synthetic construct
AEDTAVYYCAR

SEQ ID NO: 73 - protein, 14 aa, synthetic construct
AGLQAFFQVQECNK

SEQ ID NO: 74 - protein, 15 aa, synthetic construct
ITEVALMGHLSCDTK

SEQ ID NO: 75 - protein, 17 aa, synthetic construct
TCPKPDDLPFSTVVPLK

SEQ ID NO: 76 - protein, 11 aa, synthetic construct
DYELLCLDGTR

SEQ ID NO: 77 - protein, 13 aa, synthetic construct
DVMQGTDEHVVCK

SEQ ID NO: 78 - protein, 19 aa, synthetic construct
WQQGNIFSCSVMHEALHNR

SEQ ID NO: 79 - protein, 11 aa, synthetic construct
LICQATGFSPR

SEQ ID NO: 80 - protein, 11 aa, synthetic construct
MTVTDQVNCPK

SEQ ID NO: 81 - protein, 19 aa, synthetic construct
ATTVTGTPCQDWAAQEPHR

SEQ ID NO: 82 - protein, 11 aa, synthetic construct
ACEPGVDYVYK

SEQ ID NO: 83 - protein, 23 aa, synthetic construct
LVRPEVDVMCTAFHDNEETFLKK

SEQ ID NO: 84 - protein, 12 aa, synthetic construct
GPSVFPLAPCSR

SEQ ID NO: 85 - protein, 14 aa, synthetic construct
STSESTAALGCLVK

SEQ ID NO: 86 - protein, 12 aa, synthetic construct
VTAAPQSVCALR

SEQ ID NO: 87 - protein, 17 aa, synthetic construct
MCPQLQQYEMHGPEGLR

SEQ ID NO: 88 - protein, 13 aa, synthetic construct
KPVEEYANCHLAR

SEQ ID NO: 89 - protein, 32 aa, synthetic construct
SGNTFRPEVHLLPPPSEELALNELVTLTCLAR

SEQ ID NO: 90 - protein, 10 aa, synthetic construct
YDVENCLANK

SEQ ID NO: 91 - protein, 19 aa, synthetic construct
TPEVTCVVVDVSHEDPEVK

SEQ ID NO: 92 - protein, 13 aa, synthetic construct
QEPERNECFLQHK

SEQ ID NO: 93 - protein, 16 aa, synthetic construct
VELLHNPAFCSLATTK

SEQ ID NO: 94 - protein, 20 aa, synthetic construct
GDTFSCMVGHEALPLAFTQK

SEQ ID NO: 95 - protein, 6 aa, synthetic construct
CPLMVK

SEQ ID NO: 96 - protein, 22 aa, synthetic construct
ETTFNSLLCPSGGEVSEELSLK

SEQ ID NO: 97 - protein, 15 aa, synthetic construct
DCHLAQVPSHTVVAR

SEQ ID NO: 98 - protein, 21 aa, synthetic construct
TYCSEPEKVDKDNEDFQESNR

SEQ ID NO: 99 - protein, 19 aa, synthetic construct
EFNAETFTFHADICTLSEK

SEQ ID NO: 100 - protein, 27 aa, synthetic construct
YNILPEKEEFPFALGVQTLPQTCDEPK

SEQ ID NO: 101 - protein, 15 aa, synthetic construct
NYCGLPGEYWLGNDK

SEQ ID NO: 102 - protein, 24 aa, synthetic construct
EVGTPHGIILDSVDAAFICPGSSR

SEQ ID NO: 103 - protein, 14 aa, synthetic construct
WYNLAIGSTCPWLK

SEQ ID NO: 104 - protein, 16 aa, synthetic construct
SGTASVVCLLNNFYPR

SEQ ID NO: 105 - protein, 11 aa, synthetic construct
SEDCFILDHGK

SEQ ID NO: 106 - protein, 21 aa, synthetic construct
KGDTFSCMVGHEALPLAFTQK

SEQ ID NO: 107 - protein, 13 aa, synthetic construct
GTFATLSELHCDK

SEQ ID NO: 108 - protein, 18 aa, synthetic construct
EEEASSPGLGCSKPHLEK

SEQ ID NO: 109 - protein, 14 aa, synthetic construct
ADDKETCFAEEGKK

SEQ ID NO: 110 - protein, 9 aa, synthetic construct
SCGLHQLLR

SEQ ID NO: 111 - protein, 12 aa, synthetic construct
TPLGDTTHTCPR

SEQ ID NO: 112 - protein, 7 aa, synthetic construct
ECEEIIR

SEQ ID NO: 113 - protein, 7 aa, synthetic construct
AACLLPK

SEQ ID NO: 114 - protein, 13 aa, synthetic construct
TCVADESAENCDK

SEQ ID NO: 115 - protein, 21 aa, synthetic construct
DALENIDPATQMMILNCIYFK

SEQ ID NO: 116 - protein, 20 aa, synthetic construct
PEVDVMCTAFHDNEETFLKK

SEQ ID NO: 117 - protein, 19 aa, synthetic construct
ATTVTGTPCQDWAAQEPHR

SEQ ID NO: 118 - protein, 12 aa, synthetic construct
AYLEEECPATLR

SEQ ID NO: 119 - protein, 16 aa, synthetic construct
RPCFSALEVDETYVPK

SEQ ID NO: 120 - protein, 26 aa, synthetic construct
REDFDVEAADSAGNCLDSLVFVAGDR

SEQ ID NO: 121 - protein, 15 aa, synthetic construct
VCNYVNWIQQTIAAN

SEQ ID NO: 122 - protein, 13 aa, synthetic construct
KPVEEYANCHLAR

SEQ ID NO: 123 - protein, 16 aa, synthetic construct
KSPQELLCGASLISDR

SEQ ID NO: 124 - protein, 17 aa, synthetic construct
TCPKPDDLPFSTVVPLK

SEQ ID NO: 125 - protein, 26 aa, synthetic construct
LPECEADDGCPKPPEIAHGYVEHSVR

SEQ ID NO: 126 - protein, 21 aa, synthetic construct
QNCELFEQLGEYKFQNALLVR

SEQ ID NO: 127 - protein, 14 aa, synthetic construct
DLYSGLIGPLIVCR

SEQ ID NO: 128 - protein, 19 aa, synthetic construct
SAGWNIPIGLLYCDLPEPR

SEQ ID NO: 129 - protein, 19 aa, synthetic construct
FWGFHDCTHQEDVAVICSG

SEQ ID NO: 130 - protein, 16 aa, synthetic construct
KFICPLTGLWPINTLK

SEQ ID NO: 131 - protein, 23 aa, synthetic construct
CPFPSRPDNGFVNYPAKPTLYYK

SEQ ID NO: 132 - protein, 15 aa, synthetic construct
FICPLTGLWPINTLK

SEQ ID NO: 133 - protein, 14 aa, synthetic construct
WYNLAIGSTCPWLK

SEQ ID NO: 134 - protein, 15 aa, synthetic construct
FICPLTGLWPINTLK

SEQ ID NO: 135 - protein, 8 aa, synthetic construct
WELCDIPR

SEQ ID NO: 136 - protein, 21 aa, synthetic construct
ALVLIAFAQYLQQCPFEDHVK

SEQ ID NO: 137 - protein, 22 aa, synthetic construct
LVRPEVDVMCTAFHDNEETFLK

SEQ ID NO: 138 - protein, 13 aa, synthetic construct
QNCELFEQLGEYK

SEQ ID NO: 139 - protein, 13 aa, synthetic construct
KPIWLSQMSCSGR

SEQ ID NO: 140 - protein, 17 aa, synthetic construct
VYACEVTHQGLSSPVTK

SEQ ID NO: 141 - protein, 8 aa, synthetic construct
ASYLDCIR

SEQ ID NO: 142 - protein, 21 aa, synthetic construct
SCVGETTESTQCEDEELEHLR

SEQ ID NO: 143 - protein, 19 aa, synthetic construct
TPEVTCVVVDVSHEDPEVK

SEQ ID NO: 144 - protein, 8 aa, synthetic construct
LCTVATLR

SEQ ID NO: 145 - protein, 9 aa, synthetic construct
KHVAEAICK

SEQ ID NO: 146 - protein, 16 aa, synthetic construct
ADGESCSASMMYQEGK

SEQ ID NO: 147 - protein, 24 aa, synthetic construct
LLQEGQALEYVCPSGFYPYPVQTR

SEQ ID NO: 148 - protein, 21 aa, synthetic construct
TPEVTCVVVDVSHEDPEVQFK

SEQ ID NO: 149 - protein, 22 aa, synthetic construct
CSLLVLENELNAELGLSGASMK

SEQ ID NO: 150 - protein, 13 aa, synthetic construct
LHSLHLEGSCLGR

SEQ ID NO: 151 - protein, 12 aa, synthetic construct
CCESASEDCMAK

SEQ ID NO: 152 - protein, 12 aa, synthetic construct
AAFTECCQAADK